(19) United States
(12) Patent Application Publication
(10) Pub. No.: US 2024/0252405 A1
(43) Pub. Date: Aug. 1, 2024

(54) PERSONAL CARE COMPOSITIONS AND PRODUCTS

(71) Applicant: Edgewell Personal Care Brands, LLC, Chesterfield, MO (US)

(72) Inventors: David Gee, New Smyrna Beach, FL (US); Cassandra Wagers, Edgewater, FL (US); Samuel Vona, Keene Valley, NY (US)

(21) Appl. No.: 18/565,804

(22) PCT Filed: Jun. 10, 2022
(86) PCT No.: PCT/US2022/072873

§ 371 Date: Nov. 30, 2023

Related U.S. Application Data
(60) Provisional application No. 63/209,464, filed on Jun. 11, 2021.

(51) Int. Cl. A61K 8/29; A61K 8/06; A61K 8/11; A61K 8/27; A61K 8/81; A61K 8/894; A61Q 9/02; A61Q 17/04

(52) U.S. Cl. CPC A61K 8/29; A61K 8/064; A61K 8/11; A61K 8/27; A61K 8/8129; A61K 8/894; A61Q 9/02; A61Q 17/04; A61K 2800/56

(57) ABSTRACT

Embodiments relate to personal care compositions, such as shave care compositions, sun care compositions, skin care compositions, or combinations thereof, which may be in the form of a loose powder, a compacted powder, an emulsion, an oil, or a gel. Embodiments further relate to personal care products including the personal care compositions such as pods, tablets, sachets, and/or refillable systems. Packaging may house personal care compositions in the form of loose powders or fluids, the pods, the tablets, and/or the sachets. For example, the packaging may be a personal care device that allows the loose powders to foam when in contact with water or may include a container that allows the loose powders, the pods, the sachets, or the tablets to dispensed and activated by a solvent for application onto keratinous substrate or hirsute surface as a foam, a lotion, a cream, an oil, or a gel.

### CROSS-REFERENCE TO RELATED APPLICATIONS

This application claims priority to U.S. Provisional Patent Application No. 63/209,464, filed on Jun. 11, 2021, the content of which is incorporated herein by reference in its entirety.

### TECHNICAL FIELD

Embodiments relate to personal care compositions, such as shave, sun care, and/or skin care compositions. Embodiments further relate to personal care products including personal care compositions, such as loose powders or emulsions encapsulated by films to form pods or by paper to form sachets, powders compacted to form tablets, and/or loose powders, tablets, sachets, or pods housed by packaging to form refillable systems.

### BACKGROUND

Technologies are available to address personal care needs. For example, shave, sun care or skin care compositions may be available in gel, cream, lotion, stick, foam, spray, and wipe formats. Such compositions may be packaged in containers that can be bulky and difficult to manage during travel or that may not maximize sustainability benefits. For example, pouches, tubes, or aerosol canisters may utilize materials that can increase recycling or upcycling complexity and costs. Traditional formats, including soluble formats, may also include ingredients incompatible with prolonged skin contact and/or that are irritating. Thus, there is considerable room for improvement to provide personal care compositions and products with enhanced performance and/or sustainability benefits.

### SUMMARY

Embodiments relate to a personal care composition. In one example, the personal care composition may be present in the form of a loose powder, a compacted powder, an emulsion, an oil, or a gel. For example, the powder may include a surfactant, a foam promoter, a thickener, a binder, a carrier, an absorbent, or combinations thereof. For example, the emulsion may include an external oil phase. The personal care composition may be a shave personal care composition, a sunscreen personal care composition, a skin care personal care composition, or combinations thereof. In one example, the skin care personal care composition is a skin sanitizer (e.g., hand sanitizer) personal care composition.

In any of the preceding embodiments, the surfactant may include a cationic surfactant, an anionic surfactant, a nonionic surfactant, an amphoteric surfactant, or combinations thereof. At least one cationic surfactant may have antimicrobial efficacy. The cationic surfactant may include, for example, alkyl dimethyl benzyl ammonium chloride present in an amount of about 0.1 wt. % to about 5.0 wt. %, based on the total weight of the personal care composition. In one example of a powder, water may be present in an amount less than about 5.0 wt. %, based on the total weight of the personal care composition. In one example of an emulsion, water may be present in an amount ≥50.0 wt. %, based on the total weight of the personal care composition.

In any of the preceding embodiments, the surfactant may include an anionic surfactant and the foam promoter may promote foam via a gas evolution reaction. For example, the anionic surfactant may include an acylisethionate, an alkyl sulfonate, an alkyl sulfoacetate, or combinations thereof. In one example, the anionic surfactant may include sodium cocoyl isethionate and sodium lauryl sufoacetate, with or without sodium C₁₂-C₁₄ olefin sulfonate. The foam promoter may include citric acid and sodium bicarbonate.

In any of the preceding embodiments, the surfactant may include a nonionic surfactant including polyethylene oxide. In one example, the polyethylene oxide may have a molecular weight of about 100,000 Daltons to about 10,000,000 Daltons.

In any of the preceding embodiments, the thickener may include a plant-based thickener. For example, the thickener may include xantham gum, konjak gum, hydroxyethylcellulose, or combinations thereof. Additionally or alternatively, the binder may include a sugar alcohol. For example, the sugar alcohol may include mannitol, sorbitol, xylitol, erythritol, isomalt, lactitol, maltitol, or combinations thereof.

In any of the preceding embodiments, the carrier may include an emollient. For example, the emollient may include an ester of salicylic acid, of a fatty acid, of benzoic acid, or combinations thereof. In one example, the emollient may include ethylhexyl salicylate, butyloctyl salicylate, ethylhexyl palmitate, C₁₂₋₁₅ alkyl benzoate, and phenethyl benzoate. Additionally or alternatively, the absorbent may include a wax, a mineral, or combinations thereof. For example, the absorbent may include paraffin, calcium silicate, and silica.

In any of the preceding embodiments, the emulsion may include a water-in-oil emulsion. Additionally or alternatively, the surfactant may include an emulsifier having a hydrophilic-lipophilic balance ≤6. For example, the emulsifier may include a sucrose ester, a sorbitan ester, a polyglyceryl ester, a silicone, or combinations thereof. In one example, the emulsifier may include lauryl PEG-8 dimethicone, cetyl PEG/PPG-10/1 dimethicone, or combinations thereof. Additionally or alternatively, the emulsion may include an internal water phase present in an amount of about 50.0 wt. % to about 90.0 wt. %, based on the total weight of the personal care composition.

In any of the preceding embodiments, the personal care composition may further include one or more film formers, sunscreen actives, anti-aging actives, anti-acne actives, insect repellants, amino acids, colorants, pigments, dyes, opacifiers, pH adjusters, fragrances, fillers, vitamins and its derivatives, preservatives, preservative boosters, chelating agents, dispersing agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anesthetics, antiseptics, skin cooling compounds, skin protectants, skin penetration enhancers, exfoliants, staining agents, depigmenting agents, hypopigmenting agents, pharmaceutical agents, spherical powders, extracts, alpha and beta hydroxy acids, retinol and its derivatives, or combinations thereof.

Embodiments further relate to a personal care product including the personal care composition in any of the preceding embodiments. For example, the personal care product may include a water-soluble film that encapsulates the personal care composition to form a pod composed of a loose powder or an emulsion. For example, the personal care product may include water-soluble paper that encapsulates the personal care composition to form a sachet composed of a loose powder. The personal care product may also include packaging to house the personal care composition in the form of the loose powder or to house the pod (e.g., composed of the loose powder, the emulsion, etc.). However, packaging may house other formats such as tablets, water-soluble films encapsulating oils or gels, water-soluble and/or biodegradable sachets encapsulating loose powders or fluids, and so on.

In any of the preceding embodiments, the water-soluble film may include a natural polymer, a synthetic polymer, or combinations thereof. For example, the synthetic polymer may include polyvinyl alcohol. Additionally or alternatively, the water-soluble film may have a thickness from about 0.025 mm to about 0.130 mm. Additionally or alternatively, the material used to form a sachet may include paper (e.g., coated, uncoated, water-soluble, etc.), plastic (e.g., compostable, biodegradable, etc.), or combinations thereof.

In any of the preceding embodiments, the packaging may include a container having a lid, a cap, or a water-insoluble film each independently being configured to allow the personal care composition, the tablet, the sachet, and/or the pod to be deposited from the container. For example, a container housing a loose powder, a tablet, a sachet, or a pod may have a lid that can be used to deposit the loose powder or the pod. In another example, the cap may have orifices that can be used to deposit a loose powder. Meanwhile, the water-insoluble film may be perforated or retracted to allow for a loose powder, a tablet, a sachet, or a pod to be deposited from a container.

In any of the preceding embodiments, the packaging may include a personal care device having one or more pockets configured to house the personal care composition. In one embodiment, the personal care composition may be in the form a loose powder that can be mixed with water to allow the personal care composition in the form of the loose powder to foam and be captured by a user for application to a keratinous substrate or hirsute surface. In one example, a loose powder may be applied to wet skin which when activated by a solvent (e.g., water) transforms in situ on the wet skin into a foam or a lotion.

Embodiments further relate to a method to make a personal care composition and/or a personal care product in any of the preceding embodiments. For example, the method may include manufacturing and/or selecting ingredients of a personal care composition and encapsulating the personal care composition with a film to form a pod. In one example, encapsulating the personal care composition may include surrounding the personal care composition with the film and heat sealing the film to enclose the personal care composition with the film. In one example, the method may include selecting ingredients of a personal care composition and compacting the personal care composition to form a tablet. In a further embodiment, the method may include selecting ingredients of a personal care composition and encapsulating the personal care composition with a paper material to form a sachet. Additionally or alternatively, the method may include selecting packaging to house the personal care composition, the tablet, the sachet, the pod, or combinations thereof.

Embodiments further relate to a method to use a personal care composition and/or a personal care product in any of the preceding embodiments. For example, the method may include contacting the personal care product, in the form of a pod or a sachet, with a solvent (e.g., water) in an effective amount to activate a soluble polymer or paper material to deposit the personal care composition on a keratinous substrate or hirsute surface. Additionally or alternatively, the method may include contacting the personal care composition, in the form of a loose powder, or a personal care product, in the form of a compacted powder tablet, with a solvent in an effective amount to activate the personal care composition for use on a keratinous substrate or hirsute surface in various forms such as a foam, a lotion, and so on.

### BRIEF DESCRIPTION OF THE DRAWINGS

The features of the disclosure believed to be novel and the elements characteristic of the invention are set forth with particularity in the appended claims. The figures are for illustration purposes only and are not drawn to scale. The disclosure itself, however, both as to organization and method of operation, can best be understood by reference to the description of the preferred embodiment(s) which follows, taken in conjunction with the accompanying drawings in which:

FIGS. 1A-1B are examples of a personal care pod product composed of a film that encapsulates a personal care composition in the form of a loose powder and an emulsion, respectively, according to embodiments;

FIGS. 2A-2B are examples of packaging to house personal care pod products according to embodiments;

FIGS. 3A-3B are examples of packaging to house personal care compositions in the form of loose powders according to embodiments;

FIG. 4 is an example of a personal care composition in the form of a loose powder that provides a lotion according to embodiments; and

FIG. 5 is a graph illustrating water resistance and broad-spectrum ultraviolet (UV) protection of personal care compositions according to embodiments.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. In this regard, the term “(and)” is an INCI designation for a mixture of listed components separated by the term. Any numerical range recited herein is intended to include all sub-ranges subsumed therein, and such ranges are understood to include each and every number and/or fraction between the stated range lower and upper values. Also, the term “about” may refer to deviations of +20% unless otherwise stated.

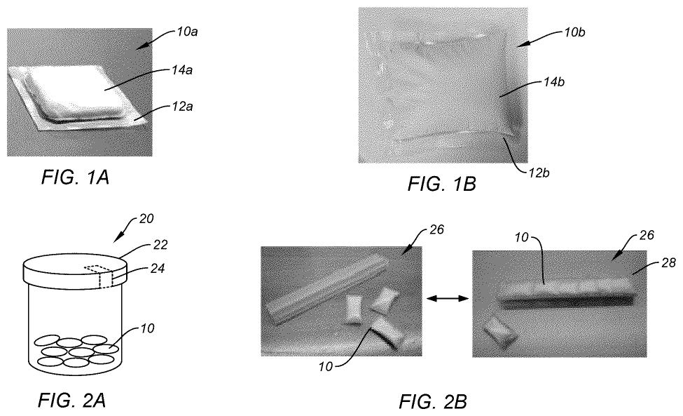

Referring to FIGS. 1A-1B, a personal care product 10 (10a-10b) is shown in a pod format including a film 12 (12a-12b) encapsulating a personal care composition (14a-14b). Other single-use or multi-use formats, however, may be implemented. For example, tablets may be formed when loose powder in an enclosed cavity of some geometry (die cavity), situated within a die that may itself be situated in a die table, has been compressed/compacted by punches. Two types of tableting machines that may be used to form a tablet include a single punch (eccentric) tablet press or a rotary tablet press. Thus, tablets may be formed by compressing loose powders using a hydraulic press, manual press, ultrasonic compaction, etc. In a further example, sachet packing machines are widely available to encapsulate powders or fluids into various types of sachets (e.g., polymer-coated paper, uncoated paper, water-soluble paper, plastic, etc.). Such sachet packing machines traditionally offer relatively fast filling and sealing for food technologies (e.g., sugar, ketchup).

Notably, pods, tablets, and sachets may be configured for single-use applications such that the personal care composition (and any encapsulating material) may be washed away during a single use. Such formats may, however, be configured for multi-use applications. For example, pods, tablets, or sachets may be dissolved by a consumer prior to a first use and then repeatedly administered from suitable packaging (e.g., pump spray, etc.) until the personal care composition has been fully exhausted or the consumer wishes to change the composition. In yet a further example, paper or plastic sachets may be used by tearing open an area of the sachet and releasing the encapsulated material (e.g., loose powders, oils, lotions, etc.). All of the material may be utilized, such as in a single use application, or some of the material may be reserved (e.g., predetermined amount, desired amount, etc.) for a later use in the multi-use application. The sachet may, in either case, be water-insoluble (e.g., biodegradable, etc.) or may be water-soluble for dissolution upon use.

Accordingly, single-use or multi-use products such as pods, tablets, or sachets may maximize sustainability, maximize portability, or provide a personal care product with configurable characteristics such as size, shape, film thickness, weight, mass/volume, performance, other functional properties, or combinations thereof. Pods, tablets, and sachets may, for example, have a circular, oval, triangular, polygonal, rectangular, square, or any shape. Pods may be composed of a film with a thickness from about 0.01 mm to about 1.0 mm, including every number and/or fraction therebetween. For example, a pod including a loose powder (e.g., a shave pod, a skin care pod such as a sanitizing pod or an anti-aging pod, a sun care pod, etc.) may be composed of a film with a thickness from about 0.025 mm to about 0.130 mm, including every number and/or fraction therebetween. Film thickness may, however, not necessarily be the same or even substantially similar in a given pod. Generally, a variable film thickness and/or pod thickness may help to maximize mechanical strength and/or solubility. In one example for shapes other than square where it might be relatively easier to cut and seal without chads or excess wasted material, for example, it may be beneficial to fold such that portions of the pod have a thicker film than other portions. Similarly, sachets may encompass a variety of materials that can be produced in multiple thicknesses, coatings, and configurations. For example, paper sachets may be stick shaped, square shaped, or custom shaped (e.g., to accommodate a solid tablet format, etc.).

Additionally, pods, tablets, and sachets may be composed of a personal care composition present at a total mass from about 0.1 mg to about 30 g or more, including every number and/or fraction therebetween. For example, a pod or a sachet may be composed of a loose powder for a shave application in an amount from about 100 mg or less to about 1.0 g or more such as, without limitation, 90 mg, 250 mg, 500 mg, 750 mg, 1.0 g, 1.1 g, including every number and/or fraction therebetween. In another example, a pod or a sachet may be composed of an emulsion or a powder (e.g., for a sunscreen application, for a skin care application, etc.) in an amount from about 3 g or more, for example about 3 g for administration to a part of a body or up to about 50 g for administration to a full body. The total mass of a personal care composition that forms a tablet may similarly be varied based one or more factors such as dissolution rate, single-use applications, multi-use applications, or combinations thereof. Thus, characteristics may be readily tailored such as dissolution rate, foaming quality, lubrication, moisturization, active agent bulk or administration amount (dosage), use environments, barrier permeability requirements, or combinations thereof.

Preferably, pods are composed of a water-soluble polymer selected from natural polymers (e.g., xanthan gum, pectins, chitosan derivatives, dextran, carrageenan, guar gum, cellulose ethers, hyaluronic acid, albumin, starch or starch derivatives), synthetic polymers (e.g., polyethyleneglycol (PEG), polyvinyl pyrrolidone (PVP), polyvinyl alcohol (PVA), polyacrylic acid (PAA), polyacrylamides, N-(2-hydroxypropyl) methacrylamide (HPMA), divinyl ethermaleicanhydride (DIVEMA), polyoxazoline, polyphosphates, polyphosphazenes), or combinations thereof. Water-soluble films are commercially available from MonoSol®, Solublon®, CHBPack®, or Ithidro®. Generally, a water-soluble polymer can encapsulate a personal care composition to form a pod by depositing the personal care composition on the water-soluble film (e.g., depositing the personal care product into a pouch formed by the film, depositing the personal care product onto the film, etc.) and sealing the personal care composition by, for example, applying pressure and/or heat to one or more areas the water-soluble film (e.g., heat sealing the pouch, wrapping the personal care composition with the same or different water-soluble film and sealing at one or more areas, etc.). As shown in FIGS. 1A-1B, the film 12 includes PVA which encapsulates the personal care composition 14.

Meanwhile, tablets may be composed of ingredients that can be compressed/compacted into a substantially solid form and dissolve or effervesce upon activation with a solvent. For example, a tablet or a portion thereof may be composed of a mixture of citric acid and sodium bicarbonate, which when exposed to water undergo chemical reaction to yield carbon dioxide and sodium citrate. Additionally or alternatively, surfactants may be implemented to aid in foaming. Accordingly, the personal care composition may be a collection of atoms or molecules held together so, under constant dry conditions, they maintain a defined shape and/or size as a solid personal care composition.

Sachets may be composed of water-insoluble material (e.g., biodegradable, etc.) or may be composed of water-soluble material for dissolution during use. For example, water-insoluble biodegradable or compostable material (e.g., plastics such as polylactic acid, coated paper, etc.) may be used to encapsulate personal care composition such as loose powders or fluids (e.g., oils, emulsions, etc.). Meanwhile, water-soluble sachets may be designed to deliver a pre-portioned product without having to open the package. Water-soluble paper products (e.g., pouches, sheets, etc.) are commercially available from, for example, SmartSolve®. While water-soluble paper sachets may also be coated, uncoated water-soluble sachets may result in relatively faster reaction time owing to the absence of films.

Notably, various packaging may be utilized to house pods, tablets, sachets, and/or free personal care compositions (e.g., loose powders, emulsions, oils, oil gels, etc.) to minimize contact with an external aqueous medium in certain embodiments. In this regard, one or more components of the packaging may be composed of materials selected to maximize sustainability such as, for example, biodegradable and/or compostable plastics, biopolymers, glass, compostable cardboard, recycled or upcycled versions of the aforementioned, or combinations thereof.

Turning now to FIG. 2A, a personal care product such as the pod 10 (FIG. 1) can be housed in a container 20 including a lid 22 that may mechanically releasably connect (e.g., threaded connection, etc.) to allow the pod 10 to be deposited from the container 20. The lid 22 may optionally include a flip-top portion 24 that stays connected when it is opened to allow the pod 10 to be deposited from the container 20. As shown in FIG. 2B, a personal care product such as the pod 10 (FIG. 1) may also be placed in a container 26 and aligned with other personal care products for storage. In the illustrated example, the container 26 has a water-insoluble film 28 but may include any film, polymer, or none at all. The water-insoluble film 28 may mechanically releasably connect (e.g., adhesive, etc.) or have portions to be traversed to access pockets that house personal care compositions and/or products. Other formats may similarly be housed in the containers 20, 26. For example, sachets (e.g., water-soluble packets) may be additionally or alternatively be housed in the containers 20, 26.

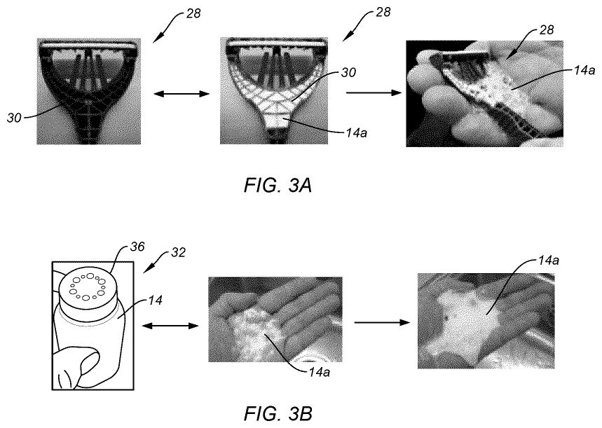

Referring to FIG. 3A, a personal care composition such as the personal care composition 14a (FIG. 1) may be present in a personal care device 28 as a loose powder. While the personal care device 28 shown is a razor, any personal care device (e.g., toothbrush, dermaplaning wand, etc.) may house the personal care composition 14a. In the example, the personal care composition 14a is disposed in a plurality of pockets 30 of the personal care device 28. The personal care composition 14a may, however, be disposed in a single pocket of the personal care device 28. Thus, a user may move the personal care device 28 to water or vice versa and catch the personal care composition 14a when it is converted to a foam. Alternatively, the personal care composition 14a may be applied to a keratinous substrate or hirsute surface in powder form and then be activated when contacted with water. In either case, FIG. 3A illustrates that the personal care device 28 may be loaded and/or re-loaded with the personal care composition 14a as needed to maximize sustainability, repeated use, etc.

As shown in FIG. 3B, the personal care composition 14 may be housed in a container 32 that allows the loose powder to be deposited on a keratinous substrate or hirsute surface. In the illustrated example, the container 32 includes a rotatable cap 34 having orifices 36 which when opened allow the personal care composition 14 to be deposited on a keratinous substrate. The container 32 may also be used to load and/or re-load a personal care device such as the personal care device 28 (FIG. 3A) discussed above. Thus, in one example, the personal care composition 14a may dissolve and/or foam during a shave when activated by water and rubbed and/or spread on a relatively large an area (e.g., face, etc.) to be shaved. Similarly, a sun care or a skin care personal care composition in the form of a loose powder may be dispensed onto on a relatively large area (e.g., face, arm, whole-body, etc.) that is to be protected (e.g., UV, repellant, etc.), treated (e.g., anti-aging, moisturized, etc.), and so on. While not shown, loose powders may also be housed in other suitable containers for multi-use applications. For example, loose powders may be placed into a pump container and filled with water for repeated administration of the personal care composition.

Referring back to FIGS. 1A-1B, the personal care compositions 14a, 14b are present in the form of a loose powder (e.g., shave pods, sun care pods, skin care pods, etc.) and an emulsion (e.g., sun care pods, skin care pods, etc.), respectively. Personal care compositions may, however, be present in various forms such as, without limitation, compacted powders, gels, oils, lotions, creams, and the like. Personal care compositions may, therefore, be composed of one or more carriers including, without limitation, water, vegetable oils, esters (e.g., octyl palmitate, isopropyl myristate, isopropyl palmitate, etc.), ethers (e.g., dicapryl ether, dimethyl isosorbide, etc.), alcohols (e.g., ethanol, isopropanol, etc.), fatty alcohols (e.g., cetyl alcohol, stearyl alcohol, behenyl alcohol, etc.), isoparaffins (e.g., isooctane, isododecane, isohexadecane, etc.), silicone oils (e.g., dimethicones, polysiloxanes, etc.), hydrocarbon oils (e.g., mineral oil, petrolatum, isoeicosane polyisobutene, etc.), polyols (e.g., propylene glycol, glycerin, butylene glycol, pentylene glycol, hexylene glycol, etc.), natural-based micelles (e.g., oleosomes), or combinations thereof.

For example, personal care compositions may be composed of one or more carriers that act as skin-conditioning agents (e.g., emollients, humectants, etc.) for moisturization, etc.

Examples of useful skin-conditioning agents include, without limitation, esters (e.g., isopropyl myristate, isopropyl palmitate, 1-decene polymer (hydrogenated), esters of benzoic acid such as lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, etc.), polyols (e.g., glycerin, butylene glycol, hexylene glycol, 2,3-butanediol, 1,2-alkane diols such propylene glycol, 1,2-butanediol, pentylene glycol, ethyl hexanediol, caprylyl glycol, decylene glycol, etc.), natural oils (e.g., nut oil, seed oil, fruit oil, etc.), fatty acids (e.g., lanolin, petrolatum, mineral oil, etc.), oleates (e.g., butyl oleate, etc.), adipates (e.g., dibutyl adipate, diisobutyl adipate, diisopropyl adipate, etc.), triglycerides (caprylic/capric triglyceride, etc.), silicones (cyclomethicone, dimethicone, etc.), salicylates (e.g., butyloctyl salicylate, C₁₂-C₁₅ alkyl salicylate, capryloyl salicylic acid, hexydodecyl salicylate, isocetyl salicylate, isodecyl salicylate, tridecyl salicylate, etc.), or combinations thereof.

Accordingly, in one example, an oil gel may be formulated using mineral oil and a gelling agent (e.g., hydrogenated butylene/ethylene/styrene copolymer, hydrogenated ethylene/propylene/styrene copolymer, etc.) present in an amount of about 0.1 wt. % to about 5.0 wt. %, based on a total weight of the personal care composition. Thickeners, discussed in further detail below, may also be used as gelling agents in hydrous or anhydrous personal care compositions. Moreover, non-aqueous carriers may be used without gelling agents for various applications. For example, mineral oil may be present in an amount of 50.0 wt. % or greater (e.g., >90 wt. %), based on a total weight of a sunscreen composition. Additionally, oils may form oil-in-oil or oil-in-silicon emulsions (e.g., castor oil in silicone oil, etc.) to deliver water-insoluble active ingredients (e.g., sunscreen actives, insect repellants, skin care actives, etc.) to a keratinous substrate. Similarly, isoparaffins are useful non-aqueous carriers. For example, isododecane or isohexadecane may be present in an amount of about 10 wt. % to about 80 wt. %, based on a total weight of a personal care composition.

Water may also be intentionally added in a personal care composition at an amount greater than 15.0 wt. % such as, for example, 25.0 wt. %, 45.0 wt. %, 55.0 wt. %, 65.0 wt. %, 75.0 wt. %, including every number and/or fraction therebetween, based on the total weight of a hydrous personal care composition. For example, a water-in-oil (W/O) emulsion may have an aqueous dispersed phase stabilized by an emulsifier and disposed in a hydrophobic continuous phase which is encapsulated in a water-soluble film. Thus, a relatively large amount of water may surprisingly be utilized in a pod together with emulsifiers that are effective to stabilize the water phase in the oil phase despite the presence of a water-soluble encapsulating film. For example, a hydrous personal care composition encapsulated in a PVA film may include ≥50.0 wt. % of water in the dispersed phase that is present in an oil phase including caprylic/capric triglyceride, butyloctyl salicylate, dimethicone, glycerine, isohexadecane, and/or PEG-8 and that is stabilized by an emulsifier (e.g., having a HLB of less than 6, lauryl PEG-8 dimethicone, cetyl PEG/PPG-10/1 dimethicone, etc.), or combinations thereof. It is understood that other ingredients, such as those discussed in detail herein and variations as falling within the true scope and spirit of the present disclosure, may be useful in embodiments (e.g., in one or more emulsion phases, etc.). For example, a W/O emulsion may include up to 90% of an internal phase of water using a non-ionic W/O emulsifier in the oil phase such as octyldodecanol (and) octyldodecyl xyloside (and) PEG-30 dipolyhydroxystearate together with stabilizers in the water phase such as polyacrylate-13 (and) polyisobutene (and) polysorbate 20 and hydroxyethyl acrylate/sodium acryloyldimethyl taurate copolymer.

Water may, however, be intentionally added in an amount of 15.0 wt. % or less, based on a total weight of the personal care composition, to form a substantially anhydrous personal care composition. Moreover, one or more ingredients may have associated with it absorbed and/or adsorbed water that can be carried into the otherwise substantially anhydrous personal care composition. For economic, sourcing, and/or formulation reasons, it may be disadvantageous to process hygroscopic materials to make them completely free of water. Thus, a substantially anhydrous personal care composition may include 15.0 wt. % or less, preferably 10.0 wt. % or less, of water. For example, a foam composition may include a surfactant and 15.0 wt. % or less, preferably 5.0 wt. % or less (e.g., ≤1.0 wt. %), of water based on a total weight of the foam composition. Similarly, a cream composition may include a surfactant, an emulsifier, and water in an amount of 15.0 wt. % or less, based on a total weight of the cream composition. In one example, a personal care composition is free of water.

Carriers may be present from about 0.1 wt. % to about 99.0 wt. %, based on the total weight of the personal care composition. A carrier may, for example, be present in an amount of 0.008 wt. %, 0.1 wt. %, 1.0 wt. %, 10.0 wt. %, 20.0 wt. %, 40.0 wt. %, 60.0 wt. %, 90.0 wt. %, 99.0 wt. %, including every number and/or fraction therebetween, based on the total weight of the personal care composition. For example, petrolatum may be present in an amount of about 20.0 wt. % to about 40.0 wt. %, C₁₂-C₁₅ alkyl benzoate may be present in an amount of about 5.0 wt. % to about 10.0 wt. %, and/or glycerin may be present in an amount of about 1.0 to about 60.0 wt. %, based on the total weight of the personal care composition.

Meanwhile, surfactants useful in embodiments may include anionic, cationic, nonionic, amphoteric, or combinations thereof. Examples of useful anionic surfactants include, without limitation, carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. For example, anionic surfactants may include salts of fatty acid esters (e.g., having from 12 to 20 carbon atoms, such as sodium methyl cocoyl taurate), salts of fatty acid isethionates (e.g., having from 12 to 20 carbon atoms such as sodium cocoyl isethionate), salts of fatty acid sulfoacetates (e.g., having from 12 to 20 carbons such as sodium lauryl sulfoacetate, disalts of fatty acid sulfosuccinates (e.g., having from 12 to 20 carbons such as disodium laureth sulfosuccinate), or combinations thereof. Thus, for example, a personal care composition may include anionic surfactants selected from salts of acylisethionate (e.g., sodium cocoyl isethionate), alkyl sulfonates (e.g., sodium C₁₂-C₁₄ olefin sulfonate, sodium cocomonoglyceride sulfonate, sodium C₁₂-C₁₅ pareth-15 sulfonate), alkyl sulfoacetates (e.g., sodium lauryl sulfoacetate), or combinations thereof.

Examples of useful cationic surfactants include, without limitation, alkylamines, alkylimidazolines, quaternary ammonium compounds (QAC), or combinations thereof. For example, cationic surfactants may include benzethonium, benzalkonium, cetrimide, cetylpyridinium chloride, stearalkonium chloride, dicetyldimonium choride, alkyl glucoside, cocoamide monoethanolamine, cocoamide, or combinations thereof. Notably, a salt of benzethonium or benzalkonium may also act as a biocide to kill a microorganism such as, for example, a virus, bacteria, or yeast. Thus, effective amounts of a benzyl substituted QAC such as alkyl dimethyl benzyl ammonium chloride (ADBAC) and/or diisobutylphenoxyethyoxyethyl dimethyl benzyl ammonium chloride may provide antimicrobial efficacy (e.g., ≥2-log kill rate, ≥3-log reduction of at least one bacteria, etc.). Generally, antimicrobial efficacy of an ingredient for a given target may be determined using applicable or known standards such as, without limitation, NF EN 1276—Biological products—Bactericidal: Antiseptics and Chemical Disinfectants; JIS Z 2801: Antimicrobial Testing of Plastics and Materials—Antimicrobial Products—Testing for Antimicrobial Activity and Efficacy; ISO 22196: Antimicrobial Testing of Plastics and Materials—Plastics—Measurement of Antibacterial Action on Plastic Surfaces.

A personal care composition may include, for example, about 0.1 wt. % to about 5.0 wt. % of one or more of the following: alkyl dimethyl benzyl ammonium chloride (benzalkonium chloride) including one or more of C₁₂ (5%) C₁₄ (60%) C₁₆ (30%) C₁₈ (5%), C₁₂ (40%) C₁₄ (50%) C₁₆ (10%), C₁₂ (50%) C₁₄ (30%) C₁₆ (17%) C₁₈ (3%), C₁₂ (1%) C₁₄ (98%) C₁₆ (1%), C₁₂ (5%) C₁₄ (60%) C₁₆ (30%) C₁₈ (5%), C₁₂ (25%) C₁₄ (60%) C₁₆ (15%), C₈₋₁₀ (2.5%) C₁₄ (61%) C₁₆ (23%) C₁₈ (2.5%), C₁₂ (14%) C₁₄ (58%) C₁₆ (28%), C₁₂ (65%) C₁₄ (25%) C₁₆ (10%), C₁₂ (67%) C₁₄ (25%) C₁₆ (7%) C₁₈ (1%), C₁₂ (61%) C₁₄ (23%) C₁₆ (11%) C₁₈ (5%), C₁₂ (3%) C₁₄ (95%) C₁₆ (2%), C₁₂ (70%) C₁₄ (30%), C₁₂ (5%) C₁₄ (90%) C₁₆ (5%); ADBA Saccharinate including C₁₂ (40%) C₁₄ (50%) C₁₆ (10%); alkyl dimethyl ethylbenzyl ammonium chloride (ADEBAC) including C₁₂ (68%) C₁₄ (32%), C₁₂ (50%) C₁₄ (30%) C₁₆ (17%) C₁₈ (3%); n-alkyl dimethyl 1-naphtylmethyl ammonium chloride including C₁₂ (98%) C₁₄ (2%); n-alkyl dimethyl dimethyl ammonium chloride including C₁₂ (68%) C₁₄ (32%); dodecyl benzyl trimethyl ammonium chloride; diisobutylphenoxyethyoxyethyl dimethyl benzyl ammonium chloride (Benzethonium Chloride); methyl dodecyl benzyl tri methyl ammonium chloride (80%) methyl dodecyl xylene bis tri methyl ammonium chloride (20%); diisobutyl cresoxyethoxyethyl dimethyl benzyl ammonium chloride monohydrate.

Examples of useful nonionic surfactants include, without limitation, alkanolamides, amine oxides, esterified carboxylic acids, ethoxylated alcohols, poloxamers, or combinations thereof. Nonionic surfactants may include alkyl glucoside, cocoamide monoethanolamine, cocoamide diethanolamine, a glycerol alkyl ester, polyethylene glycol, poloxamer 337, poloxamer 407, or combinations thereof. In one example, polyethylene oxide (PEO) that is commercially available as POLYOX™ from The Dow Chemical Company in a variety of molecular weights and particle sizes can be used. As known in the industry and references in POLYOX™ commercial literature, the molecular weight of POLYOX™ polyethylene oxides, such as those, are determined largely by viscosity data. Thus, one or more PEOs may be selected based on a molecular weight (MW) of about 100,000 Daltons (Da) to about 10,000,000 Da, for example to about 5,000,000 Da. For example, PEO or a portion of PEO in a personal care composition may include higher molecular weight PEO (e.g., 2,000,000 Da or more), while in other embodiments all or a portion of PEO in a personal care composition may include lower molecular weight PEO (e.g., less than 2,000,000 Da). In one example, PEO includes PEG-115M commercially available as POLYOX™ WSR Coagulant.

Examples of useful amphoteric/zwitterionic surfactants include, without limitation, betaines, sultaines, cocamphoacetates, or combinations thereof. For example, an amphoteric surfactant may be selected from fatty aminopropionates (e.g., characterized with the general formula R—NH—(CH₂)₂—COONa), fatty iminodipropionates (e.g., characterized with the general formula R—N—((CH₂)₂—COONa)₂), alkyl betaines (e.g., alkyl dimethyl betaines characterized with the general formula R—N—(CH₃)₂—CH₂—COO), alkyl amidopropyldimethylbetaines (e.g., characterized with the general formula R—CO—NH—(CH₂)₃—N—(CH₃)₂—CH₂—COO), imidazoline derived amphoterics, alkyl poly amino carboxylates (e.g., characterized by four or more nitrogen atoms and four or more carboxylic acid groups), or combinations thereof. Thus, a personal care composition may include amphoteric surfactants selected from cocamidopropyl betaine, lauramidopropyl betaine, cocoamido hydroxyl sultaine, sodium cocamphoacetate, sodium methyl cocoyl taurate, or combinations thereof.

Examples of emulsifiers, which are surfactants that stabilize emulsions, may include W/O emulsifiers, oil-in-water (O/W) emulsifiers, or combinations thereof. A W/O emulsifier may have a hydrophilic-lipophilic balance (HLB) of ≤6, such as from 4 to 6. Examples of useful W/O emulsifiers in embodiments include, without limitation, certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), glycerin and/or fatty acid esters (e.g., glyceryl stearate, etc.), or combinations thereof. Meanwhile, a O/W emulsifier may have HLB≥7, such as 7 to 18. Examples of O/W emulsifiers include certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB greater than 8, etc. Additionally, useful natural emulsifiers may include, without limitation, cetearyl alcohol (and) cetearyl glucoside, glyceryl stearate (and) cetearyl alcohol (and) sodium stearoyl lactylate, cetearyl wheat straw glycosides (and) cetearyl alcohol, sorbitan olivate, cetearyl olivate (and) sorbitan olivate, or combinations thereof.

Surfactants may be present from about 0.1 wt. % to about 99.0 wt. %, based on the total weight of the personal care composition. A surfactant may, for example, be present in an amount of 0.008 wt. %, 0.1 wt. %, 1.0 wt. %, 10.0 wt. %, 20.0 wt. %, 40.0 wt. %, 60.0 wt. %, 90.0 wt. %, 99.0 wt. %, including every number and/or fraction therebetween, based on the total weight of the personal care composition. For example, PEO may be present in an amount of about 0.1 wt. % to about 5.0 wt. %, based on the total weight of the personal care composition. Additionally, sodium cocoyl isethionate, sodium lauryl sulfoacetate, and/or sodium C₁₄₋₁₆ olefin sulfonate may be present in an amount of about 15.0 wt. % to about 65.0 wt. %, based on the total weight of the personal care composition.

Embodiments may include a thickener to enhance the stability of emulsions, create gels, and/or provide texture. Examples of useful thickeners include, without limitation, plant-based thickeners (e.g., xanthan gum, konjac gum, guar gum, acacia gum, cellulose derivatives such as hydroxycellulose, hydroxyethyl cellulose, etc.), mineral thickeners (e.g., silica, bentonite, magnesium aluminum silicate, etc.), lipid thickeners (e.g., cetyl alcohol, stearyl alcohol, waxes such as carnauba wax, stearic acid, etc.), synthetic thickeners (e.g., carbomer, acrylates/C₁₀-30 alkyl acrylate crosspolymer, etc.), or combinations thereof.

Thickeners may be present from about 0.1 wt. % to about 40.0 wt. %, based on the total weight of the personal care composition. A thickener may, for example, be present in an amount of 0.008 wt. %, 0.1 wt. %, 1.0 wt. %, 5.0 wt. %, 10.0 wt. %, 20.0 wt. %, 30.0 wt. %, 40.0 wt. %, including every number and/or fraction therebetween, based on the total weight of the personal care composition. For example, xanthan gum may be present in an amount of about 0.1 wt. % to about 5.0 wt. % to stabilize an emulsion. Notably, a thickener such as xanthan gum, konjac gum, etc., may be present in a powder foam composition in an amount of about 30 wt. % to about 40 wt. %, based on the total weight of the powder foam composition, to enhance foam viscosity or foam stability as well as to provide lubrication for slip and/or glide during shaving. In another example, hydroxycellulose may be present in an amount of about 5.0 wt. % to about 15 wt. %, based on the total weight of the power foam composition. In a further example, konjac gum may be present in an amount of about 10.0 wt. % to about 20.0 wt. %, based on the total weight of the power foam composition.

Embodiments may include a binder to hold ingredients together. Binders may include, for example, dry binders, wet binders, or combinations thereof. For example, dry binders may include zinc stearate, magnesium myristate, lithium stearate, or combinations thereof. Meanwhile, wet binders may include hydrocarbons, esters, silicones, or combinations thereof. Preferably, the binder includes a sugar alcohol such as, without limitations, mannitol, sorbitol, xylitol, erythritol, isomalt, lactitol, maltitol, or combinations thereof. Binders may be present from about 10.0 wt. % to about 60.0 wt. %, based on the total weight of the personal care composition. In one example where a binder is used with a thickener, the ratio of binder:thickener may be from about 1:4 or about 4:1, for example about 1:2.

Embodiments may include an absorbent to absorb an ingredient. An absorbent may include natural materials, synthetic materials, or combinations thereof. Natural materials include, without limitation, bentonite, amylodextrin, Avena Sativa, alumina, or combinations thereof. In one example, a natural absorbent material may include Copemicia Cerifera (Carnauba) wax (and) calcium silicate. Synthetic materials include, without limitation, starch octenylsuccinate, paraffin (and) calcium silicate (and) silica, or combinations thereof. A binder and/or an absorbent may, for example, be present in an amount of 9.0 wt. % wt. %, 15.0 wt. %, 22.0 wt. %, 30.0 wt. %, 35.0 wt. %, 45.0 wt. %, 60.0 wt. %, 66.0 wt. %, including every number and/or fraction therebetween, based on the total weight of the composition.

Embodiments may include a foam promoter to promote foam via a gas evolution reaction. A foam promoter may include an acid (e.g., citric acid, tartaric acid, etc.), a phosphate powder (e.g., calcium dihydrogen phosphate), a sulfate powder (e.g., sodium aluminum sulfate, etc.), or combinations thereof. A foam promoter may also include a carbonate (e.g., sodium carbonate, etc.), a bicarbonate (e.g., sodium bicarbonate, potassium bicarbonate, etc.), or combinations thereof. Thus, foam promoters may produce a gas that promotes foam when an acidic agent and an alkali agent are combined in the presence of an initiator (e.g., solvent such as water or alcohol, heat, etc.). Foam promoters may be present from about 1.0 wt. % to about 10.0 wt. %, based on the total weight of the personal care composition. A foam promoter may, for example, be present in an amount of 0.08 wt. %, 1.0 wt. %, 3.0 wt. %, 5.0 wt. %, 10.0 wt. %, including every number and/or fraction therebetween, based on the total weight of the personal care composition. For example, citric acid and sodium bicarbonate may together be present in an amount of about 5.0 wt. % to about 10.0 wt. %, based on the total weight of the personal care composition.

Embodiments may include a film former to provide a substantially homogenous and/or structured hydro-lipidic film on skin owing to its viscosity-building properties. Useful film formers may be synthesized from two or more different monomers to form copolymers and/or from individual polymer chains that are connected by bridging molecules or crosslinking agents to form crosslinked copolymers known as crosspolymers. Film formers may be present from about 0.1 wt. % to about 5.0 wt. %, based on the total weight of the personal care composition. A film former may, for example, be present in an amount of 0.08 wt. %, 1.0 wt. %, 2.0 wt. %, 3.0 wt. %, 4.0 wt. %, 5.0 wt. %, 5.5 wt. %, including every number and/or fraction therebetween, based on the total weight of the personal care composition.

Useful film formers may include, without limitation, monomers, copolymers, cross polymers, and/or terpolymers of an organo-silicone hybrid, biopolymer, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. For example, a film former may include crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, methyl dihydroabietate, trimethylsiloxysilicate, acrylate/C₁₂-C₂₂ (meth)acrylate copolymer, C₂₄-C₂₈ alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, shellac, stearyl/octyldodecyl citrate crosspolymer, or combinations thereof.

Embodiments may include other ingredients such as, without limitation, sunscreen actives, anti-aging actives, anti-acne actives, insect repellants, amino acids, colorants, pigments, dyes, opacifiers, pH adjusters, fragrances, fillers, vitamins (e.g., A, C, E, and/or derivatives), preservatives, preservative boosters, chelating agents, dispersing agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anesthetics, antiseptics, skin cooling compounds, skin protectants, skin penetration enhancers, exfoliants, staining agents, depigmenting agents, hypopigmenting agents, pharmaceutical agents, spherical powders, extracts (fruit, flower, plant), alpha and beta hydroxy acids, retinol and its derivatives, or combinations thereof.

For example, embodiments may include a sunscreen active to substantially absorb, scatter, and/or deflect ultraviolet (UV) radiation on a keratinous substrate such as, without limitation, a p-aminobenzoic acid, a benzophenone, a camphor, a cinnamate, a dibenzoylmethane, a salicylate, an imidazole, a triazole, a triazine, a triazole, triazone, a metal oxide, or combinations thereof. Thus, for example, the sunscreen composition may include one or more organic sunscreen actives such as: p-aminobenzoic acids such as p-aminobenzoic acid (CAS #: 150-13-0), p-aminobenzoic acid, monoglyceryl ester (CAS #: 136-44-7), p-aminobenzoic acid, octyl dimethyl ester (padimate-0, CAS #: 21245-02-3), p-aminobenzoic acid, ethyl dihydroxypropyl (roxadimate, CAS #: 58882-17-0), p-aminobenzoic acid, 2,4-dihydroxy-N-(3-hydroxypropyl)-3,3-dimethylbutanamide ester, p-aminobenzoic acid, PEG-25 (CAS #: 116242-27-4); anthranilates such as menthyl anthranilate (CAS #: 134-09-8); benzophenones such as benzophenone (CAS #: 119-61-9), 2,4-dihydroxybenzophenone (benzophenone-1, CAS #: 131-56-6), 2,2′,4,4′-tetrahydroxybenzophenone (benzophenone-2, CAS #: 131-55-5), 2-hydroxy-4-methoxybenzophenone (benzophenone-3, oxybenzone, CAS #: 131-57-7), 2-hydroxy-4-methoxybenzophenone-5-sulfonic acid (benzophenone-4, sulisobenzone, CAS #: 4065-45-6), 2-hydroxy-4-methoxybenzophenone-5-sulfonic acid, monosodium salt (benzophenone-5, CAS #: 6628-37-1), 2,2′-dihydroxy-4,4′-dimethoxybenzophenone (benzophenone-6, CAS #: 131-54-4), 5-chloro-2-hydroxybenzophenone (benzophenone-7, CAS #: 85-19-8), 2,2′-dihydroxy-4-methoxybenzophenone (benzophenone-8, dioxybenzone, CAS #: 131-53-3), 2,2′-dihydroxy-4,4′-dimethoxybenzophenone-5,5′-disulfonic acid sodium salt (benzophenone-9, CAS #: 76656-36-5), 2-hydroxy-4-methoxy-4′-methyl-benzophenone (benzophenone-10, Mexenone, CAS #: 1641-17-4), bis(2,4-ihydroxyphenyl)methanone (benzophenone-11, CAS #: 1341-54-4), 2-hydroxy-4-(octyloxy)benzophenone (benzophenone-12, octabenzone, CAS #: 1843-05-6), 2,2′-dihydroxy-4-methoxybenzophenone (dioxybenzone, CAS #: 131-53-3), 2-hydroxy-4-methoxybenzophenone (oxybenzone, CAS #: 131-57-7), 2-hydroxy-4-methoxybenzophenone-5-sulphonic acid (sulisobenzone, CAS #: 4065-45-6), hexyl 2-(4-(diethylamino)-2-hydroxybenzoyl)benzoate (CAS #: 302776-68-7); camphors such as benzylidene camphor sulfonic acid (CAS #: 56039-58-8), 3-benzylidene camphor (CAS #: 15087-24-8), polyacrylamidomethyl benzylidene camphor (CAS #: 113783-61-2), terephthalylidene dicamphor sulfonic acid (CAS #: 90457-82-2), 3-(4-methylbenzyliden) camphor (CAS #: 36861-47-9), camphor benzalkonium methosulfate (CAS #: 52793-97-2), bornelone (CAS #: 2226-11-1); cinnamates such as ethyl cinnamate (CAS #: 103-36-6), 2-ethoxyethyl-p-methoxycinnamate (cinoxate, CAS #: 104-28-9), isoamyl p-methoxycinnamate (CAS #: 71617-10-2), diisopropyl methyl cinnamate (CAS #: 32580-71-5), 2-ethylhexyl alpha-cyano-beta-phenylcinnamate (octocrylene, CAS #: 6197-30-4), diethanolamine methoxycinnamate (CAS #: 56265-46-4), isopropyl methoxycinnamate (CAS #: 5466-76-2), isoamyl p-methoxycinnamate (CAS #: 71617-10-2), glyceryl octanoate dimethoxycinnamate, ethyl diisopropylcinnamate (CAS #: 32580-72-6), ethyl methoxycinnamate (CAS #: 99880-64-5), octyl methoxycinnamate (octinoxate, CAS #: 5466-77-3); dibenzoylmethanes such as dibenzoylmethane (CAS #: 120-46-7), isopropyl dibenzoylmethane (eusolex, CAS #: 63250-25-9), 4-tert-Butyl-4′-methoxy-dibenzoylmethane (avobenzone, CAS #: 70356-09-1); salicylates such as 3,3,5-trimethylcyclohexyl salicylate (homosalate, CAS #: 118-56-9), benzyl salicylate (CAS #: 118-58-1), octyl salicylate (octisalate, CAS #: 118-60-5), 2-hydroxyethyl salicylate (CAS #: 87-28-5), menthyl salicylate (CAS #: 89-46-3), isopropylbenzyl salicylate (CAS #: 94134-93-7); imidazoles such as phenylbenzimidazole, phenylbenzimidazol-5-sulfonic acid (CAS #: 27503-81-7), phenylbenzimidazole tea sulfonate (CAS #: 73705-00-7), urocanic acid [4-imidazoleacrylic acid, CAS #: 104-98-3], ethyl urocanate (CAS #: 27538-35-8), bisymidazylate (CAS #: 180898-37-7), sodium phenylbenzimidazole sulfonate (CAS #: 5997-53-5); triazines such as bis(ethylhexyloxyphenol methoxyphenol) triazine (bemotrizinol, CAS #: 187393-00-6); triazoles such as 2-(2-hydroxy-5-methyl-phenyl)benzotriazole (drometrizole, CAS #: 2440-22-4); triazones such as bis-ethylhexyloxyphenol methoxyphenyl triazine (iscotrizinol, CAS #: 154702-15-5), ethylhexyl triazone (CAS #: 88122-99-0); petrolatum jelly (CAS #: 8009-03-8), acrylates such as diurethane dimethacrylate (CAS #: 103597-45-1), ethyl 2-cyano-3,3-diphenylacrylate (etocrilene, CAS #: 5232-99-5); siloxanes such as drometrizole trisiloxane (CAS #: 155633-54-8); or combinations thereof. Preferably, a sunscreen composition provides protection against UVA and/or UVB radiation. For example, the sun protection factor (SPF) of a sunscreen composition may be greater than or equal to 8 and the UVA protection factor (UVAPF) may be ⅓ the SPF. A sunscreen active may also provide protection against damaging radiation within other regions in the electromagnetic spectrum such as, for example, blue light (380 nm to 500 nm) or infrared (700 nm to 1 mm). Accordingly, embodiments may include a sunscreen active that protects against UVA radiation such as avobenzone (e.g., Parasol® 1789), diethylamino hydroxybenzoyl hexyl benzoate (e.g., Uvinul® A Plus), terephthalylidene dicamphor sulfonic acid, bis-disulizole disodium, disodium phenyl dibenzimidazole tetrasulfonate, bis-diethylamino hydroxybenzoyl benzoate, bis-benzoxazolylphenyl ethylhexylamino triazine, or combinations thereof. Additionally or alternatively, embodiments may include a sunscreen active that protects against UVB radiation such as octocrylene, octinoxate, octisalate, homosalate, ensulizole, ethylhexyl triazone (e.g., Uvinul® T150), enzacamene, amiloxate, bemotrizinol (e.g., Uvasorb® HEB), benzylidene malonate polysiloxane, padimate-O, trolamine salicylate, cinoxate, PABA, or combinations thereof. Additionally or alternatively, embodiments may include an organic sunscreen active that protects against UVA and UVB radiation such as oxybenzone, meradimate, bis-octrizole, bis-ethylhexyloxyphenol methoxyphenyl triazine (Tinosorb® S), drometrizole trisiloxane, sulisobenzone, dioxybenzone, or combinations thereof. Moreover, a sunscreen active that provides protection against UVA, UVB, VIS, and IR radiation may include a physical sunscreen active as titanium dioxide, zinc oxide, or combinations thereof, which can be used in effective amounts to provide an SPF of 8 or more (in contrast to other uses including as colorants, etc.). Such filters may have various morphologies such as crystalline form (e.g., rutile, etc.), size (e.g., nano, non-nano, etc.), shape (e.g., amorphous, spherical, etc.), and functionalization (e.g., coatings, etc.) that allow for the effective use as actives. For example, titanium dioxide may have a coating of polymers (e.g., silicone copolymers, etc.), fatty acids, salts of fatty acids, oxides of silicon, oxides of aluminum, oxides of zirconium, or combinations thereof.

In a further example, embodiments may include antioxidants to counter free radicals that damage biomolecules. Antioxidants may be obtained, for example, from the extracts of flowers, fruits, vegetables, nuts, berries, plants, or combinations thereof. Antioxidants may include polyphenols and catechin derivatives such as catechin, epigallocatechin, epicatechin, epicatechin gallate, epigallocatechin gallate, gallocatechin gallate, gallocatechin, or combinations thereof. Such constituents are effective scavengers of reactive oxygen species and may also function indirectly as antioxidants through their effects on transcription factors and enzyme activities. Additionally or alternatively, antioxidants may include carotenoids (e.g., beta-carotene, lycopene, lutein, zeaxanthin), additional polyphenols (e.g., resveratrol), selenium, vitamins (A, B, C, E), grape seed extract, green tea extract, Coenzyme Q10, derivatives thereof, or combinations thereof. Meanwhile, other non-limiting examples of skin care actives that provide skin benefits may include, for example, anti-acne ingredients, anti-aging ingredients, exfoliation ingredients, or combinations thereof. Anti-acne actives may include, without limitation, benzoyl peroxide, salicylic acid, retinoids (e.g., adapalene, tretinoin, etc.), azelaic acid, antibiotics (clindamycin, erythromycin, sulfacetamide, etc.), sulfur ingredients, or combinations thereof. Anti-aging ingredients may include retinol, bakuchiol, or combinations thereof. For example, a skin care composition may include bakuchiol to improve skin tone, improve skin elasticity, smooth fine lines, smooth wrinkles, or combinations thereof. Bakuchiol may preferably be present with an oil-based skin conditioning agent selected from hemp oil, bisabolol, Adansonia digitata seed oil, or combinations thereof. Meanwhile, exfoliation particles may include any material suitable to exfoliate the skin such as, without limitation, fruit seeds, fruit stones, nut shells, ground or fibrous plant material, polymers (e.g., natural, synthetic, etc.), mineral composites, or combinations thereof. Chemical exfoliants may additionally or alternatively be used.

Auxiliary or other ingredients may be present in an amount of about 0.001 wt. % to about 99.0 wt. %, based on the total weight of the personal care composition. An auxiliary ingredient may, for example, be present in an amount of 0.0008 wt. %, 0.1 wt. %, 0.4 wt. %, 1.0 wt. %, 5.0 wt. %, 10.0 wt. %, 20.0 wt. %, 50.0 wt. %, 90.0 wt. %, 99.0 wt. %, including every number and/or fraction therebetween, based on the total weight of the personal care composition. For example, a sunscreen active may be present in an amount of about 1.0 wt. % to about 40.0 wt. %, based on the total weight of the sunscreen composition. In another example, a skin care active (e.g., anti-aging, etc.) may be present in an amount of about 0.001 wt. % to about 40.0 wt. %, based on the total weight of the personal skin care composition.

In the following examples, loose powders are encapsulated by a water-soluble film to form a pod. It is understood that the loose powders may, alternatively, be compacted to form tablets and/or may be further formulated to have any ingredients discussed in detail herein as well as variations falling within the true scope and spirit of the present disclosure to obtain desirable pod or tablet characteristics such as size, shape, color, dissolution rate, foaming quality, lubrication, moisturization, active agent dosage, or combinations thereof. Similarly, loose powders and fluids (e.g., oils, emulsions, etc.) may be packaged in other suitable consumer product packaging such as tubes, bottles, tottles, etc. Moreover, loose powders and fluids may be encapsulated by sachets (e.g., water-soluble paper sachet, biodegradable plastics, etc.) and delivered with or without packaging (e.g., a container, etc.).

### Example Shave Compositions and Products

Example Table 1 shows embodiments of a shave composition in the form of a powder. The ingredients were added in a single phase according to traditional processes know in the art. Example formulations, having the ingredients shown in Table 1 within the listed 5 approximate wt. %, were encapsulated in PVA films to form pods.

TABLE I
Example Shave Powders

| Component (INCI) | Function | Approx. wt. % |
|---|---|---|
| PEO, Sodium Cocoyl Isethionate, | Surfactant | 90.0-95.0 |
| Sodium Lauryl Sulfoacetate, Sodium | | |
| C₁₂₋₁₄ olefin sulfonate | | |
| Citric Acid, Sodium Bicarbonate | Foam Promoter | 5.0-10.0 |
| Titanium Dioxide (and) Zinc Stearate | Opacifier | 0.0-1.0 |

Example Table 2 shows embodiments of a shave composition in the form of a powder. The ingredients were added in a single phase according to traditional processes know in the art. Example formulations, having the ingredients shown in Table 2 within the listed approximate wt. %, were encapsulated in PVA films to form pods.

TABLE II
Example Shave Powders

| Component (INCI) | Function | Approx. wt. % |
|---|---|---|
| Sodium Cocoyl Isethionate, Sodium | Surfactant | 50.0-65.0 |
| Lauryl Sulfoacetate, Sodium C₁₂₋₁₄ | | |
| olefin sulfonate | | |
| Xanthan Gum | Thickener | 30.0-40.0 |
| Citric Acid, Sodium Bicarbonate | Foam Promoter | 5.0-10.0 |
| Titanium Dioxide (and) Zinc Stearate | Opacifier | 0.0-1.0 |

Example Table 3 shows an embodiment of a shave composition in the form of a powder. The ingredients were added in a single phase according to traditional processes know in the art. Example formulations, having the ingredients shown in Table 3 within the 5 listed approximate wt. %, were encapsulated in PVA films.

TABLE III
Example Shave Powders

| Component (INCI) | Function | Approx. wt. % |
|---|---|---|
| Sodium Cocoyl Isethionate, Sodium | Surfactant | 40.0-50.0 |
| Lauryl Sulfoacetate | | |
| Xanthan Gum, Konjac Gum | Thickener | 40.0-50.0 |
| Citric Acid, Sodium Bicarbonate | Foam Promoter | 5.0-10.0 |
| Magnesium Carbonate | Opacifier | 0.1-1.5 |

Example Table 4 shows an embodiment of a shave composition in the form of a powder. The ingredients were added in a single phase according to traditional processes know in the art. An example formulation, having the ingredients shown in Table 4 within the listed approximate wt. %, was encapsulated in a PVA film to form a pod.

TABLE IV
Example Shave Powders

| Component (INCI) | Function | Approx. wt. % |
|---|---|---|
| PEO, Sodium Cocoyl Isethionate, | Surfactant | 80.0-90.0 |
| Sodium Lauryl Sulfoacetate | | |
| Hydroxyethylcellulose | Thickener | 7.5-15.0 |
| Citric Acid, Sodium Bicarbonate | Foam Promoter | 5.0-10.0 |
| Titanium Dioxide (and) Zinc Stearate | Opacifier | 0.1-1.5 |

Example Table 5 shows an embodiment of a shave composition in the form of a powder. The ingredients were added in a single phase according to traditional processes know in the art. In one example formulation, the ingredients shown in Table 5 within the listed approximate wt. % were placed in powder form into various containers, discussed in more detail above, and dispended and/or contacted with water to foam in the presence of running water (e.g., running water, potable water, etc.).

TABLE V
Example Shave Powders

| Component (INCI) | Function | Approx. wt. % |
|---|---|---|
| PEO, Sodium Cocoyl Isethionate | Surfactant | 10.0-20.0 |
| Citric Acid, Sodium Bicarbonate | Foam Promoter | 80.0-90.0 |

Descriptive sensory analysis was conducted on various shave formulations described above using panelists trained to detect and quantify sensations related to shave performance. This type of evaluation can be very useful in identifying and tracking specific changes among samples. Moreover, data collected is statistically robust. Example evaluations are as follows.

Analysis 1: Example formulations having the ingredients, minus opacifier, shown in Table 2 (present within the listed approximate wt. %) were encapsulated in PVA films to form pods and tested on twenty-nine panelists. Overall the panelists had positive comments about how quick and easy the pods dissolved, shave performance, and the rinse off skin. Ratings after three shaves on a seven-point scale were recorded as follows: Ease of Dissolving Pod (4.24); Ease to Spread (3.94); Ease to Lather (3.64); Reduces Dryness (4.99); Soft Skin (5.00); Smooth Skin (5.06); Moisturized Skin (4.97); Skin Rinsability (4.46); Razor Rinsability (4.54); Reduces Shave Irritation (4.74); Safe from Nicks and Cuts (5.03); Comfortable Shave (4.70); Provides Close Shave (4.82); Prevents Pulling and Tugging (5.09); Improves Razor Glide (5.01).

Analysis 2: A comparison was made on twenty-six panelists between example formulations from Analysis 1, except now including opacifier and about 50% more payload compared to the formulations of Analysis 1, and formulations having the ingredients shown in Table 1 within the listed approximate wt. %, also having opacifier and at the same payload quantity (i.e., 50% more payload compared to the formulations in Analysis 1). The thickness of the film remained the same between the pods of Analysis 1 and Analysis 2. After two shaves, the combined shave results indicate that the later formulations rated significantly higher than the former formulations for Overall Liking but there was not a significant difference for Overall Performance. The later formulations were rated significantly higher than the former formulations for Rinse off Skin and Rinse off Razor. The later formulations were also rated directionally higher than the former formulations for Ease of Dissolving Pod and Ease to Spread. The former formulations scored directionally higher for Reduce Shave Irritation. All remaining performance attributes showed no significant difference.

Analysis 3: A comparison was made on thirty panelists between example formulations having the ingredients shown in Table 2 from Analysis 2 and formulations having the ingredients shown in Table 3 within the listed approximate wt. %, also having opacifier and at the same payload quantity. The thickness of the film remained the same. After two shaves, the combined shave results indicate no significant difference for Overall Liking. The former formulations rated significantly higher than the later formulations for Overall Performance, Ease to Lather, Tracking, Close Shave, Prevents Pulling/Tugging, Reduces Redness, and Smooth Skin. The former formulations also scored directionally higher for Ease to Spread. All remaining performance attributes showed no significant difference.

### Example Sun Care Compositions and Products

Example Table 6 shows an embodiment of sunscreen compositions in the form of a W/O emulsion. The ingredients were added in phases according to traditional processes known in the art. Example formulations, having the ingredients shown in Table 6 within the listed approximate wt. %, were encapsulated in a PVA film. The encapsulated example formulations included approximate sunscreen active: carrier/oil phase: emulsifier: carrier/dispersed phase of 1:4:3:10, and exhibited no film degradation for over a month.

TABLE VI
Example Sunscreen W/O Emulsion

| | | Approx. |
|---|---|---|
| Component (INCI) | Function | wt. % |
| Example Main Ingredients | | |
| Zinc Oxide, Titanium Dioxide | Sunscreen Active | 10.0-15.0 |
| (and) Alumina (and) Dimethicone | | |
| Caprylic/Capric Triglyceride, | Carrier/Oil Phase/ | 20.0-25.0 |
| Butyloctyl Salicylate, Dimethicone, | External Phase | |
| Glycerine, Isohexadecane, PEG-8 | | |
| Lauryl PEG-8 Dimethicone, Cetyl | Emulsifier | 2.0-7.5 |
| PEG/PPG-10/1 Dimethicone | | |
| Water | Carrier/Dispersed | 50.0-80.0 |
| | Phase/Internal Phase | |
| Example Additional Ingredients | | |
| VP/Eicosene Copolymer, | Film Former, | 0.01-6.5 |
| Polyhydroxystearic acid, Ethylhexyl | Thickener, Photo- | |
| Methoxycrylene, Phenoxyethanol, | stabilizer/Booster, | |
| Caprylyl Glycol, Sodium Chloride, | Preservative System, | |
| Sodium Citrate, Tocopheryl Acetate, | pH Adjuster, Vitamin, | |
| pigment, and/or odorant | Colorant, Fragrance | |

Notably, the samples included water present in an amount over 50.0 wt. %, for example from about 50.0 wt. % to about 60.0 wt. %, and were encapsulated by a heat sealed PVA film. The samples, however, did not rupture at room temperature or at 50° ° C. after four weeks. While W/O emulsions are listed in Table 6, multiple formats can be utilized, including oils, water-in-silicone emulsions, oil-in-silicone, silicone-in-oil emulsions, and multiple 10 emulsions (e.g., O/W/O, etc.). In this regard, substantially anhydrous formulas, or emulsions having an anhydrous external phase, may be relatively stable and/or may provide relatively more accurate dosing (e.g., pods can be sized by intended use—face, arms, legs, full body, etc.), reduce relative waste (e.g., less HDPE or other plastic containers), and/or provide sustainable packaging (e.g., PVA is water soluble and biodegradable).

While the formulations in Table 6 include sunscreen actives it is understood than any auxiliary or active ingredient may be utilized such as, without limitation, skin care anti-aging actives (e.g., hyaluronic acid, vitamins, retinol, etc.), biocides (e.g., ADBACs, etc.), insect repellants (e.g., lemon eucalyptus oil, etc.), or combinations thereof. For example, benzalkonium chloride or other active (e.g., dispersed in water) may be present in an aqueous discontinuous phase of an emulsion and encapsulated by a water-soluble film (e.g., PVA, etc.). Similarly, water or oil soluble actives (e.g., anti or pro microbial, anti-wrinkle, anti-age spot, anti-pollution, anti-insect, etc.) may be present in an appropriate phase of an emulsion and/or more generally in a carrier, and encapsulated by a water-soluble film (e.g., PVA, etc.).

Example Table 7 shows an embodiment of sunscreen compositions in the form of a powder. The ingredients were added in phases according to traditional processes know in the art. Example formulations, having the ingredients shown in Table 7 within the listed approximate wt. %, were placed into containers for storage and deposition.

TABLE VII
Example Sunscreen Powder

| | | Approx. |
|---|---|---|
| Component (INCI) | Function | wt. % |
| Octocrylene, Homosalate, | Sunscreen Active | 20.0-25.0 |
| Avobenzone | | |
| Ethylhexyl Salicylate, Butyloctyl | Carrier/Oil Phase | 40.0-50.0 |
| Salicylate, Ethylhexyl Palmitate, C₁₂₋₁₅ | | |
| Alkyl Benzoate, Phenethyl Benzoate | | |
| Paraffin (and) Calcium Silicate (and) | Absorbent | 25.0-35.0 |
| Silica | | |

The example formulations were substantially anhydrous, having less than about 5.0 wt. % of water, preferably less than about 1.0 wt. % of water. The example formulations include a powder composed of an oil phase containing emollients and/or active ingredients in synthetic oil absorbent powder having a wax and a mineral. Example compositions may, however, be formulated with additional or alternative absorbents such as, for example, a natural oil absorbent powder having a wax and a mineral. Similarly, sunscreen compositions may be formulated with any sunscreen active such as, for example, octisalate in addition to or as an alternative to homosolate, BEMT, metal oxides, and so on.

While the formulas in Table 7 include sunscreen actives it is understood than any auxiliary or active ingredient may be utilized such as, without limitation, skin care actives (e.g., hyaluronic acid, vitamins, retinol, etc.), insect repellants (e.g., lemon eucalyptus oil, etc.), or combinations thereof. For example, substantially anhydrous actives may be present in a powder and encapsulated by a water-soluble film (e.g., PVA, etc.). Similarly, water or oil soluble skin actives (e.g., ani-wrinkle, anti-age spot, anti-pollution, anti-insect, etc.) may be present in a powder and encapsulated by a water-soluble film (e.g., PVA, etc.).

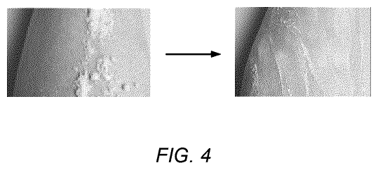

As shown in FIG. 4, the example formulations may be applied to wet skin to form a lotion for application or reapplication of sunscreen actives. The example formulations may, however, also be mixed with water to form a lotion before application (e.g., as a loose powder, in a pod, etc.). The powdered lotion may advantageously reduce reapplication time by substantially or completely eliminating the need to dry skin before reapplication. In addition, the powdered lotion may provide a relatively light-weight or sustainable alternative when compared to carrying a lotion having water and/or traditional packaging.

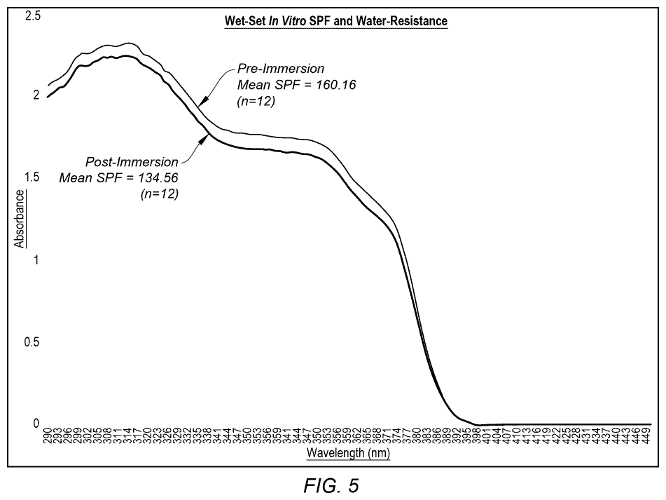

As shown in FIG. 5, the example formulations may provide relatively good water resistance, may be formulated to provide any desired SPF value (including relatively high SPF values as shown), and/or may substantially maintain SPF values post-emersion. In vitro data may be readily obtained using a Labsphere Ultraviolet Transmittance Analyzer (Model UV-2000 available from the Solar Light Company, Philadelphia, Pennsylvania). The readings may be recorded by the analyzer and the calculation of the SPF values can be based on the following equation:

$$SPF = \frac{\text{?} {E}_{λ} \cdot {S}_{λ} \cdot d λ}{\text{?} {E}_{λ} \cdot {S}_{λ} \cdot {T}_{λ} \cdot d λ}$$

$$\text{?} \text{indicates text missing or illegible when filed}$$

with built-in software: UV-2000 application Version 1.1.0.0, wherein E_{λ} is the Commission Internationale de l'Eclairage (CIE) erythemal spectral effectiveness, S_{λ} is designated as the solar spectral irradiance, and T_{λ} is the spectral transmittance of the sample as measured on the UV-2000.

### Example Skin Care Compositions

Additional example skin care compositions may be formulated to include any ingredients discussed in detail herein as well as variations falling within the true scope and spirit of the present disclosure. For example, one or more of the example formulations discussed above in Tables 1-7 may be modified to include active ingredients such as, for example, skin care actives (e.g., hyaluronic acid, vitamins, retinol, etc.), biocides (e.g., ADBACs, etc.), insect repellants (e.g., lemon eucalyptus oil, etc.), or combinations thereof. In one example, benzalkonoium chloride may be present in an amount of about 0.1 wt. % to about 5.0, for example, 0.08 wt. %, 0.1 wt. %, 1.0 wt. %, 2.5 wt. %, 5.2 wt. %, including every number and/or fraction therebetween, based on the total weight of a personal care composition. Notably, the form of the additional ingredients may be selected based on a given format (e.g., dispersed forms in aqueous or non-aqueous carriers, anhydrous salt forms, pods, powders, sachets, tablets, etc.). Moreover, one or more ingredients may also be readily omitted or added based on the format, packaging, function, and so on.

For example, the anionic surfactants shown in the formulations of Table 4 may be replaced with one or more surfactants (e.g., nonionic, cationic, and/or amphoteric surfactants) and packaged together with an active such as skin care actives (e.g., hyaluronic acid, vitamins, retinol, etc.), biocides (e.g., ADBACs, etc.), insect repellants (e.g., lemon eucalyptus oil, etc.). In one example, PEO may be the only surfactant or may be present with one or more other nonionic surfactants, alone or in combination with a cationic surfactant and/or an amphoteric surfactant. One or more other ingredients may also be omitted or added, such as optionally including the foam promoter and opacifier shown in Table 4 and/or optionally including one or more ingredients discussed in detail herein as well as variations falling within the true scope and spirit of the present disclosure. In another example, one or more sunscreen actives shown in Table 7 may be replaced in whole or in part by one or more ingredients (e.g., benzalkonium chloride, anti-aging ingredients, antioxidants, etc.) provided that the other ingredients shown in Table 7 and/or the additional ingredients (e.g., actives, carriers, surfactants, etc.) account for concentration differences between additional ingredients used and sunscreen actives reduced or removed.

In yet another example, benzalkonium chloride or other active (in dispersed or salt form) may be added into the W/O formulations of Table 6. One or more other ingredients may also be omitted or added, for example additionally or alternatively using alkyl dimethicone copolyol W/O emulsifiers selected from silicone alkyl polyethers (e.g., cetyl PEG/PPG-10/1 dimethicone, lauryl PEG/PPG-18/18 dimethicone, lauryl PEG-8 dimethicone, etc.) silicone polyethers (e.g., PEG/PPG-18/18 dimethicone, PPG-8 Dimethicone, etc.), natural emulsifiers, or combinations thereof. Similarly, one or more sunscreen actives shown in Table 6 may be replaced in whole or in part by one or more ingredients (e.g., benzalkonium chloride, anti-aging ingredients, antioxidants, etc.) provided that the other ingredients shown in Table 6 and/or additional ingredients (e.g., actives, carriers, surfactants, etc.) account for concentration differences between additional ingredients used and sunscreen actives reduced or removed.

### ADDITIONAL NOTES AND EXAMPLES

Additional examples include a composition, a packaged product including a composition, a method of making a composition, and/or a method of using a composition.

Example 1 may include a personal care product including a personal care composition in the form of a loose powder, a compacted powder, an emulsion, an oil, or a gel. The personal care product of Example 1 may include a pod, a tablet, a sachet, a container, or combinations thereof. Example 1 may additionally or alternatively consist of the personal care composition, independently from the personal care product.

Example 2 may include the personal care product of Example 1, wherein the personal care composition is a shave personal care composition, such as a shave preparation composition, a sun care personal care composition, such as a sunscreen composition, a skin care personal care composition, such as a skin sanitizer composition, or combinations thereof.

Example 3 may include the personal care product of any of Examples 1 to 2, wherein the personal care product comprises a personal care composition in the form of a loose powder, a compacted powder, or an emulsion, the powder comprising a surfactant, a foam promoter, a thickener, a binder, a carrier, an absorbent, or combinations thereof, the emulsion comprising an external oil phase, and the personal care product comprising a water-soluble film to form a pod, water-soluble paper to form a sachet, or packaging to house the personal care composition, the pod, the sachet, the tablet, or combinations thereof.

Example 4 may include the personal care product of any of Examples 1 to 3, wherein the personal care composition is in the form of a loose powder or an emulsion, wherein the loose powder comprises a surfactant, a foam promoter, a thickener, a binder, a carrier, an absorbent, or combinations thereof, wherein the emulsion comprises an external oil phase, and wherein the personal care composition is a shave composition, a sunscreen composition, or a hand sanitizer composition, and wherein the personal care product further comprises a water-soluble film that encapsulates the personal care composition to form a pod or wherein the personal care product further comprises packaging to house the personal care composition in the form of the loose powder, the pod, or combinations thereof.

Example 5 may include the personal care product of any of Examples 1 to 4, wherein the surfactant comprises a cationic surfactant, a nonionic surfactant, an amphoteric surfactant, or combinations thereof, and wherein at least one cationic surfactant has an antimicrobial efficacy.

Example 6 may include the personal care product of any of Examples 1 to 5, wherein the at least one cationic surfactant is present in an amount of about 0.1 wt. % to about 5.0 wt. %, based on the total weight of the personal care composition.

Example 7 may include the personal care product of any of Examples 1 to 6, wherein the cationic surfactant comprises alkyl dimethyl benzyl ammonium chloride.

Example 8 may include the personal care product of any of Examples 1 to 7, further comprising water present in an amount of less than about 5.0 wt. %, based on the total weight of the personal care composition.

Example 9 may include the personal care product of any of Examples 1 to 8, wherein the surfactant comprises an anionic surfactant, and wherein the foam promoter promotes foam via a gas evolution reaction.

Example 10 may include the personal care product of any of Examples 1 to 9, wherein the anionic surfactant comprises an acylisethionate, an alkyl sulfonate, an alkyl sulfoacetate, or combinations thereof.

Example 11 may include the personal care product of any of Examples 1 to 10, wherein the anionic surfactant comprises sodium cocoyl isethionate and sodium lauryl sufoacetate.

Example 12 may include the personal care product of any of Examples 1 to 11, wherein the anionic surfactant further comprises sodium C₁₂-C₁₄ olefin sulfonate.

Example 13 may include the personal care product of any of Examples 1 to 12, further comprising a nonionic surfactant.

Example 14 may include the personal care product of any of Examples 1 to 13, wherein the nonionic surfactant comprises polyethylene oxide having a molecular weight of about 100,000 Daltons to about 10,000,000 Daltons.

Example 15 may include the personal care product of any of Examples 1 to 14, wherein the thickener comprises a plant-based thickener.

Example 16 may include the personal care product of any of Examples 1 to 15, wherein the plant-based thickener comprises xantham gum, konjak gum, hydroxyethylcellulose, or combinations thereof.

Example 17 may include the personal care product of any of Examples 1 to 16, wherein the binder comprises a sugar alcohol including mannitol, sorbitol, xylitol, erythritol, isomalt, lactitol, maltitol, or combinations thereof.

Example 18 may include the personal care product of any of Examples 1 to 17, wherein the foam promoter comprises citric acid and sodium bicarbonate.

Example 19 may include the personal care product of any of Examples 1 to 18, wherein the carrier comprises an emollient.

Example 20 may include the personal care product of any of Examples 1 to 19, wherein the emollient comprises an ester of salicylic acid, an ester of a fatty acid, an ester of benzoic acid, or combinations thereof.

Example 21 may include the personal care product of any of Examples 1 to 20, wherein the emollient comprises ethylhexyl salicylate, butyloctyl salicylate, ethylhexyl palmitate, C₁₂₋₁₅ alkyl benzoate, and phenethyl benzoate.

Example 22 may include the personal care product of any of Examples 1 to 21, wherein

the absorbent comprises a wax, a mineral, or combinations thereof.

Example 23 may include the personal care product of any of Examples 1 to 22, wherein the absorbent comprises paraffin, calcium silicate, and silica.

Example 24 may include the personal care product of any of Examples 1 to 23, wherein the emulsion comprises a water-in-oil emulsion.

Example 25 may include the personal care product of any of Examples 1 to 24, wherein the surfactant comprises an emulsifier having a hydrophilic-lipophilic balance ≤6.

Example 26 may include the personal care product of any of Examples 1 to 25, wherein the emulsifier comprises a sucrose ester, a sorbitan ester, a polyglyceryl ester, a silicone, or combinations thereof.

Example 27 may include the personal care product of any of Examples 1 to 26, wherein the emulsifier comprises lauryl PEG-8 dimethicone, cetyl PEG/PPG-10/1 dimethicone, or combinations thereof.

Example 28 may include the personal care product of any of Examples 1 to 27, wherein the emulsion comprises an internal water phase present in an amount of about 50.0 wt. % to about 90.0 wt. %, based on the total weight of the personal care composition.

Example 29 may include the personal care product of any of Examples 1 to 28, wherein the water-soluble film comprises a natural polymer, a synthetic polymer, or combinations thereof.

Example 30 may include the personal care product of any of Examples 1 to 29, wherein the synthetic polymer comprises polyvinyl alcohol.

Example 31 may include the personal care product of any of Examples 1 to 30, wherein the water-soluble film has a thickness from about 0.025 mm to about 0.130 mm.

Example 32 may include the personal care product of any of Examples 1 to 31, wherein the packaging comprises a container including a lid, a cap, a pump spray, or a water-insoluble film configured to allow one or more of the personal care composition or the pod to be deposited from the container.

Example 33 may include the personal care product of any of Examples 1 to 32, wherein the packaging comprises a personal care device having one or more pockets configured to house the personal care composition, wherein the personal care composition is in the form of the loose powder.

Example 34 may include the personal care product of any of Examples 1 to 33, further comprising one or more film formers, sunscreen actives, anti-aging actives, insect repellants, amino acids, colorants, pigments, dyes, opacifiers, pH adjusters, fragrances, fillers, vitamins and its derivatives, preservatives, preservative boosters, chelating agents, dispersing agents, stabilizers, sphingolipids, antioxidants, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin protectants, penetration enhancers, exfoliants, staining agents, depigmenting agents, hypopigmenting agents, pharmaceutical agents, spherical powders, extracts, alpha and beta hydroxy acids, retinol and its derivatives, or combinations thereof.

Example 35 may include a method to use any of the personal care products or personal care compositions of any of Examples 1 to 34.

Example 36 may include a method to manufacture any of the personal care products or personal care compositions of any of Examples 1 to 34.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also, the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

1-20. (canceled)

21. A personal care product comprising:
a personal care composition in the form of an emulsion, wherein the emulsion comprises an external oil phase, wherein the emulsion comprises an internal water phase present in an amount of about 50.0 wt. % to about 90.0 wt. %, based on the total weight of the personal care composition, and wherein the emulsion is a fluid; and
a water-soluble film that encapsulates the personal care composition to form a pod.

22. The personal care product of claim 21, wherein the personal care composition comprises a cationic surfactant.

23. The personal care product of claim 22, wherein the cationic surfactant is present in an amount of about 0.1 wt. % to about 5.0 wt. %, based on the total weight of the personal care composition.

24. The personal care product of claim 23, wherein the cationic surfactant comprises alkyl dimethyl benzyl ammonium chloride.

25. The personal care product of claim 21, wherein the emulsion comprises an emulsifier including a sucrose ester, a sorbitan ester, a polyglyceryl ester, a silicone, a glycerin ester, a fatty acid ester, or combinations thereof.

26. The personal care product of claim 21, wherein the emulsion comprises an emulsifier including lauryl PEG-8 dimethicone, cetyl PEG/PPG-10/1 dimethicone, or combinations thereof.

27. The personal care product of claim 21, wherein the emulsion comprises an emulsifier having a hydrophilic-lipophilic balance of less than or equal to 6.

28. The personal care product of claim 21, wherein the external oil phase comprises caprylic/capric triglyceride, butyloctyl salicylate, dimethicone, glycerine, isohexadecane, PEG-8, or combinations thereof.

29. The personal care product of claim 21, wherein the external oil phase comprises a non-ionic water-in-oil emulsifier, and wherein the internal water phase comprises a stabilizer including polyacrylate-13 (and) polyisobutene (and) polysorbate 20 and hydroxyethyl acrylate/sodium acryloyldimethyl taurate copolymer.

30. The personal care product of claim 21, wherein the water-soluble film comprises a natural polymer, a synthetic polymer, or combinations thereof.

31. The personal care product of claim 30, wherein the synthetic polymer comprises polyvinyl alcohol.

32. The personal care product of claim 21, wherein the water-soluble film has a thickness from about 0.025 mm to about 0.130 mm.

33. The personal care product of claim 21, wherein the personal care composition is present in an amount from about 3 g to about 50 g.

34. The personal care product of claim 21, wherein the personal care composition comprises a sunscreen active present in an amount sufficient to provide the personal care composition with a sun protection factor (SPF) greater than or equal to 8.

35. The personal care product of claim 34, wherein the ratio of sunscreen active:external oil phase:emulsifier:internal water phase is about 1:4:3:10.

36. The personal care product of claim 21, further including packaging to house the pod.

37. A personal care product comprising:
a personal care composition in the form of an emulsion, wherein the emulsion comprises a hydrophobic continuous phase, wherein the emulsion comprises an aqueous dispersed phase present in an amount of about 50.0 wt. % to about 90.0 wt. %, based on the total weight of the personal care composition, and wherein the emulsion is a fluid; and
a water-soluble film that encapsulates the personal care composition to form a pod.

38. The personal care product of claim 37, wherein the emulsion comprises a water-in-oil emulsion, a water-in-silicone emulsion, a multiple emulsion, or combinations thereof.

39. The personal care product of claim 37, wherein the personal care composition comprises an emulsifier disposed in the hydrophobic continuous phase.
